(19) United States
(12) Patent Application Publication
(10) Pub. No.: US 2014/0121356 A1
(43) Pub. Date: May 1, 2014

(54) HYPOALLERGENIC MUTANT POLYPEPTIDES BASED ON FISH PARVALBUMIN

(71) Applicant: BIOMAY PRODUKTIONS- UND HANDELS-AKTIENGESELLSCHAFT, Vienna (AT)

(72) Inventors: Rudolf VALENTA, Theresienfeld (AT); Peter Valent, Vienna (AT); Susanne Spitzauer, Vienna (AT); Ines Swoboda, Vienna (AT)

(73) Assignee: BIOMAY PRODUKTIONS- UND HANDELS-AKTIENGESELLSCHAFT, Vienna (AT)

(21) Appl. No.: 14/137,341

(22) Filed: Dec. 20, 2013

Related U.S. Application Data
(63) Continuation of application No. 12/721,090, filed on Mar. 10, 2010, now abandoned.
Application No. 12/721,090 is a continuation-in-part of application No. 10/924,200, filed on Aug. 24, 2004, now Pat. No. 7,696,314.

(30) Foreign Application Priority Data
Sep. 4, 2003 (EP) 03020063.8

(51) Int. Cl. C07K 14/76

(52) U.S. Cl. CPC C07K 14/76
USPC 530/350

(57) ABSTRACT

The present invention relates to non-naturally occurring polypeptides derived from fish allergens such as parvalbumin Cyp c 1.01 from carp. The polypeptides display reduced allergenic activity and are useful as allergy vaccines for treatment of sensitized allergic patients and for prophylactic vaccination.

### CROSS-REFERENCE TO RELATED APPLICATIONS

This is a Continuation-in-part of U.S. patent application Ser. No. 10/924,200, filed Aug. 24, 2004 and claiming the benefit of priority from European Application No. 03.02006.8, filed Sep. 4, 2003, each of which is hereby incorporated by reference in their entirety.

### SEQUENCE LISTING

The instant application contains a Sequence Listing which has been submitted via EFS-Web and is hereby incorporated by reference in its entirety. Said ASCII copy, created on Mar. 5, 2010, is named 02936800.txt, and is 36,210 bytes in size.

### BACKGROUND OF THE INVENTION

The present invention relates to polypeptides derived from the major fish parvalbumins. The polypeptides display reduced allergenic activity and are useful as allergy vaccines for treatment of sensitized allergic patients and for prophylactic vaccination.

Together with milk, egg, peanuts, tree nuts and shellfish, fish represents the most important source of allergens in the induction of IgE mediated food hypersensitivity (Bischoff et al., 1996; Etesamifar and Wüthrich 1998; Sampson 1999). Although not a common health problem on a world wide basis, fish allergy can reach a prevalence of 1 per 1000 individuals in fish-eating and fish-processing countries (Aas 1987). In sensitised individuals contact with and consumption of fish, but also inhalation of vapour generated during cooking can cause a variety of IgE-mediated clinical symptoms affecting the skin, the respiratory tract and the gastrointestinal tract (Aas 1987; Pascual et al., 1992; O'Neil et al., 1993).

Paravalbumins, small calcium-binding proteins, highly abundant in the white muscles of lower vertebrates (Pechere 1997) and present in lower amounts in fast twitch muscles of higher vertebrates (Lehky et al., 1974), were identified as the major fish allergens. These proteins belong to the EF-hand superfamily of calcium-binding proteins, which is characterised by the presence of helix-loop-helix metal-binding domains, termed EF-hands (Kretsinger, 1980). Parvalbumins contain three such EF-hand motifs, (AB, CD and EF sites) (Berchtold, 1989; Heizmann and Hunziker, 1991; Ikura, 1996). Two of the sites (CD and EF) are paired to form a stable domain capable of binding two cations, Ca²⁺ or Mg²⁺. The first site (AB) is unable to bind cations, but forms a cap that covers the hydrophobic surface of the pair of functional domains and thereby acts as a stabilising element (Kretsinger and Nockold, 1973; Declercq et al., 1991; Permyakov et al., 1991). Based on amino acid sequence data the parvalbumin protein family can be subdivided into two evolutionary distinct lineages: the α group, consisting of less acidic parvalbumins with isoelectric points at or above pl 5.0 and the β group, consisting of more acidic parvalbumins with isoelectric points at or below pl 4.5 (Goodman and Pechere, 1977).

Parvalbumins show a remarkable resistance to heat, denaturing chemicals and proteolytic enzymes, characteristics, which might enable them to act as potent sensitising agents for more than 95% of fish allergic patients (Aas and Elsayed, 1969; De Martino et al., 1990; O'Neil et al., 1993; Lindstroem et al., 1996; Bugajska-Schretter et al., 1998).

The inventors found that patients who mount IgE antibodies against parvalbumin of one fish species also recognise the homologous proteins from other fish species (Bugajska-Schretter et al., 1998). In IgE competition experiments performed with purified carp parvalbumin they further showed that carp parvalbumin contains the majority of IgE epitopes present in fish protein extracts of various species (Bugajska-Schretter et al., 2000). This demonstrated the importance of parvalbumins as cross-reactive fish allergens and explained why allergic individuals exhibit clinical symptoms upon contact with various fish species.

Until now, the only curative approach towards type I allergy is allergen-specific immunotherapy, which is based on the systemic administration of increasing amounts of disease-eliciting allergens in the form of allergen-containing extracts, with the aim to induce a state of allergen-specific non-responsiveness in the patient (Bousquet et al., 1998). However, although allergen-specific immunotherapy is most widely used for the treatment of respiratory- and venom allergies, it is too dangerous to be applied in case of food allergies. This is due to the extremely high risk of severe anaphylactic reactions caused by systemic application of food allergens and by the presence of several ill defined components in food extracts (and especially in fish extracts).

During the last few years the application of recombinant DNA technology into the field of Allergology has allowed to produce an increasing number of biologically active recombinant allergens by cDNA cloning and expression in heterologous hosts (Valenta and Kraft, 1995). With recombinant allergens, which closely mimic the allergenic activity of their natural counterparts, it has become possible to determine the individual patients reactivity profile and to develop component-based vaccination strategies for patient-tailored specific immunotherapy (Valenta et al., 1998).

By screening of a carp muscle cDNA expression library with serum IgE of fish allergic patients the inventors isolated cDNA clones coding for IgE-reactive parvalbumin isoforms (Cyp c 1.01 and Cyp c 1.02) and produced the first recombinant fish parvalbumin (rCyp c 1.01) with immunological features comparable to the natural allergen (Swoboda et al., 2002). rCyp c 1.01 reacted with IgE from all fish allergic patients tested, induced specific and dose-dependent basophil histamine release and contained most of the IgE epitopes (70%) present in natural allergen extracts from various fish species.

The wildtype rCyp c 1.01 molecule can not be used for therapeutical purposes. Administration, even at very low doses, would carry an enormous risk of inducing life-threatening anaphylactic side effects. One objective of the present invention is to provide mutants or variants of rCyp c 1.01 with reduced allergenic activity. The inventors propose that one possibility to obtain such hypoallergenic parvalbumin derivatives will be the site-directed mutagenesis of critical amino acids either within or outside of IgE epitopes in a way that alters the fold and decreases the secondary structure content of the protein. However, the resistance of parvalbumins to destabilising factors such as heat, denaturing chemicals or proteolytic enzymes (Elsayed and Aas, 1971) suggests that it might be difficult to obtain dramatic conformational changes, which modify the allergenic activity of the molecule, by alteration of only a few amino acids.

### BRIEF SUMMARY OF THE INVENTION

The invention aims at providing means for the prophylactic or therapeutic treatment of food allergy to fish allergens. It has been found that mutants of carp parvalbumin, e.g., of Cyp c 1.01, show strongly reduced IgE binding and are thus useful as hypoallergenic agents. The amino acid sequence of Cyp c 1.01 is shown in SEQ ID NO:1. The invention relates to a non-naturally occurring or mutated polypeptide derived from fish parvalbumin, selected from the group consisting of
a. polypeptides comprising an amino acid sequence in which in respect to the amino acid sequence as shown in SEQ ID NO:1 one to 15 amino acid residues are deleted, substituted and/or added;
b. polypeptides comprising a fragment of (a), wherein the fragment has a length of at least 15 amino acids and at least 90% of the amino acid residues of the fragment are identical to corresponding residues of the amino acid sequence as shown in SEQ ID NO:1;
c. polypeptides comprising a fragment of the amino acid sequence as shown in SEQ ID NO:1, wherein the fragment has a length of at least 15 amino acids;
d. polypeptides consisting of a fragment of (a), wherein the fragment has a length of at least 10 amino acids and at least 80% of the amino acid residues of the fragment are identical to corresponding residues of the amino acid sequence as shown in SEQ ID NO:1; and
e. polypeptides consisting of a fragment of the amino acid sequence as shown in SEQ ID NO:1, wherein the fragment has a length of at least 10 amino acids.

In another embodiment, the invention relates to an isolated fish parvalbumin polypeptide comprising an amino acid sequence that is at least 60% identical to SEQ ID NO:1 and which upon alignment of the amino acid sequence with SEQ ID NO:1, the amino acid(s) in at least two of the positions that correspond to positions 52, 54, 91 and 93 of SEQ ID NO:1, differs from the amino acids at such positions in the native amino acid sequence of the polypeptide, and wherein the fish parvalbumin polypeptide has a reduced allergenic activity compared to SEQ ID NO:1.

In another embodiment, the invention relates to an isolated fish parvalbuimin polypeptide comprising an amino acid sequence selected from the group consisting of SEQ ID NO: 7, 8, 9, 10, 11, 12, 13, 14, 15, 19, 20, 21 and 22, each of which has been mutated at amino acid residues at positions that correspond to one or more of positions 52, 54, 91 and 93 of SEQ ID NO:1, when each of said amino acid sequences is aligned with SEQ ID NO:1, wherein the mutation is a substitution of the native amino acid at such position with an amino acid that is not native to that position, and wherein said fish parvalbumin polypeptide has reduced allergenic activity compared to SEQ ID NO:1.

In another embodiment, the invention relates to an isolated fish parvalbumin polypeptide selected from the group consisting of SEQ ID NO: 2, 3, 23, 24, 25, 26, 27, 28, 29, 30, 31, 32, 33, 34, 35, 36 and 37.

In another embodiment, the invention relates to a pharmaceutical composition for the treatment or prophylaxis of an allergic reaction to fish parvalbumin, comprising at least one of SEQ ID NO: 2, 3, 23, 24, 25, 26, 27, 28, 29, 30, 31, 32, 33, 34, 35, 36 and 37 and a pharmaceutically acceptable carrier.

In another embodiment, the invention relates to an isolated polypeptide variant of SEQ ID NO: 1, wherein the isolated polypeptide variant comprises the amino acid sequence of SEQ ID NO: 1, except at least two of amino acid residues 52, 54, 91 and 93 of SEQ ID NO:1 are substituted with another amino acid and wherein the isolated polypeptide variant has less allergenic activity compared to SEQ ID NO:1.

In another embodiment, the invention relates to an isolated fish parvalbumin polypeptide variant of an amino acid sequence or a biologically active fragment of the amino acid sequence, wherein the amino acid sequence is selected from the group consisting of SEQ ID NO: 1, SEQ ID NO: 19, SEQ ID NO: 20, SEQ ID NO: 21, SEQ ID NO: 15 and SEQ ID NO: 22, except that at least two of amino acid residues 52, 54, 91 and 93 of the amino acid sequence are substituted with another amino acid and wherein the isolated polypeptide variant or biologically active fragment thereof has less allergenic activity as compared to the amino acid sequence.

In another embodiment, the invention relates to an isolated fish parvalbumin polypeptide variant which is selected from the group consisting of SEQ ID NO: 2, SEQ ID NO: 3, SEQ ID NO: 4, SEQ ID NO: 23, SEQ ID NO: 24; SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36 and SEQ ID NO: 37, or a biologically active fragment thereof. In one embodiment, such biologically active fragment comprises amino acids residues 52-95 of the variant. In another embodiment, the fragment may comprise amino acid residues 52-102, 40-102, 30-105, 20-105, 10-109 and 2-109 of said variant. In a preferred embodiment, the biologically active fragment is a fragment of SEQ ID No: 4 selected from the group consisting of amino acid residues 52-95, 52-102, 40-102, 30-105, 20-105, 10-109 and 2-109 of SEQ ID No: 4 and wherein the fragment has less allergenic activity compared to SEQ ID NO:1.

In another embodiment, the invention relates to a fusion protein comprising one of the above described fish parvalbumin polypeptides variants or biologically active fragments thereof.

In another embodiment, the invention relates to a pharmaceutical composition for the treatment or prophylaxis of an allergic reaction to fish parvalbumin, the composition comprising one or more of the above described fish parvalbumin polypeptides, variants or biologically active fragment thereof and a pharmaceutically acceptable carrier.

In another embodiment, the invention relates to a polynucleotide encoding one of the above described polypeptide, variants or biologically active fragments. In another embodiment, the invention relates to a vector or plasmid containing such polynucleotide and to host cells transformed or transfected with such vector or plasmid.

In another embodiment, the invention relates to a pharmaceutical composition for the treatment or prophylaxis of an allergic reaction to fish parvalbumin, comprising the the above described polynucleotide.

### DETAILED DESCRIPTION OF THE INVENTION

As used herein, the term “polypeptide” denotes a compound comprising at least 7 amino acids which are linked by peptide bonds. The polypeptide is preferably composed only of amino acids, but it may also comprise non-proteinaceous components. The length of the polypeptide is preferably at least 10 amino acids, more preferably at least 15 amino acids. The polypeptide may also be a fusion protein comprising a portion which is derived from Cyp c 1.01 or other fish parvalbumin (as shown in Table 1 and FIG. 7) and a fusion partner. The portion derived from Cyp c 1.01 or other fish parvalbumin may further be linked to a carrier molecule, e.g. keyhole limpet hemocyanin (KLH).

The term “fish parvalbumin” as used herein designates a calcium binding polypeptide. In one embodiment, the amino acid sequence of the “fish parvalbumin” is at least 60% identical to the amino acid sequence as shown in SEQ ID NO:1. These polypeptides have amino acid sequences identical to the respective naturally occurring fish allergens.

A “mutated” polypeptide or polypeptide “variant” according to the invention is a polypeptide which has been manipulated by introducing a mutation (substitution, addition or deletion) to a naturally occurring polypeptide such as a wild type (“WT”) fish parvalbumin.

A “non-naturally occurring” polypeptide according to the invention is a polypeptide which is structurally different from polypeptides that can be found in nature such as wild type fish parvalbumins.

An amino acid substitution denotes the replacement of one amino acid with a different amino acid. Preferably, acidic residues (glutamic acid, aspartic acid) are substituted. The substituting amino acid may be of any type, preferably it is not an acidic amino acid, more preferably it is a hydrophobic amino acid, still more preferably the substituting amino acid is selected from the group consisting of glycine, alanine, valine, isoleucine and leucine, most preferably, it is alanine.

In one embodiment, the polypeptide of the invention comprises an amino acid sequence which has 1 to 15 amino acid deletions, substitutions and/or additions in respect to the amino acid sequence as shown in SEQ ID NO: 1 or with respect to a naturally occurring sequence, e.g. the wild type sequences of FIG. 7. Preferably, the number of amino acid deletions, substitutions and/or additions is 1 to 10, more preferably 1 to 6, still more preferably 2 to 6, most preferably 2, 3 or 4. The polypeptides of this embodiment are at least about 94 amino acids in length, the preferred length is about 109 amino acids. The preferred polypeptides have 1, 2, 3, 4, 5 or 6 amino acid substitutions in respect to the amino acid sequence as shown in SEQ ID NO:1 or with respect to a naturally occurring amino acid sequence of a fish parvalbumin, such as those set forth in FIG. 7.

The one or more amino acid substitutions may include substitutions at amino acid positions 52, 54, 91 and/or 93 of SEQ ID NO:1 or from the other wild type sequence of FIG. 7. The polypeptide of the invention may comprise an amino acid sequence which has at least one amino acid substitution in respect to the amino acid sequence as shown in SEQ ID NO:1 or other wild type sequence of FIG. 7, wherein said at least one amino acid substitution is at a position selected from amino acid positions 52, 54, 91 or 93 of the sequence. The polypeptide of the invention may comprise an amino acid sequence which has at least two amino acid substitutions in respect to the amino acid sequence as shown in SEQ ID NO:1 or other wild type sequence of FIG. 7, wherein said at least two amino acid substitutions are at positions selected from amino acid positions 52, 54, 91 or 93 of the sequence. The polypeptide of the invention may comprise an amino acid sequence which has at least three amino acid substitutions in respect to the amino acid sequence as shown in SEQ ID NO:1 or other wild type sequence of FIG. 7, wherein said at least three amino acid substitutions are at positions selected from amino acid positions 52, 54, 91 or 93. The polypeptide of the invention may comprise an amino acid sequence which has at least four amino acid substitutions in respect to the amino acid sequence as shown in SEQ ID NO:1 or other wild type sequence of FIG. 7, wherein said at least four amino acid substitutions are at positions selected from amino acid positions 52, 54, 91 or 93.

The most preferred polypeptides of this aspect comprise an amino acid sequence as shown in SEQ ID NO:2, SEQ ID NO:3 or SEQ ID NO:4. The polypeptide represented by SEQ ID NO:2 carries two mutations as compared with the amino acid sequence as shown in SEQ ID NO:1, namely D52A and D54A. The polypeptide represented by SEQ ID NO:3 has the mutations D91A and D93A as compared with SEQ ID NO:1. The polypeptide represented by SEQ ID NO:4 carries mutations D52A, D54A, D91A and D93A as compared with SEQ ID NO:1. Further aspects of the invention are biologically active polypeptides comprising amino acids 52 to 93, 52 to 102, 40 to 102, 30 to 105, 20 to 105, 10 to 109 or 2 to 109 of SEQ ID NO:4. The respective lower and upper limits may be cross-combined. These amino acid positions and biologically active fragments can be applied to other fish parvalbumins as well, such as those set forth in FIG. 7.

The invention further concerns polypeptides comprising a biologically active fragment of the polypeptides described above and in Table 1 and FIG. 7. The fragment has a length of at least 15 amino acids, i.e. it consists of at least 15 consecutive amino acids of the polypeptide described above. Preferably, the length of the fragment is at least 20 amino acids, more preferably at least 25 amino acids, even more preferably at least 30 amino acids. In one embodiment, at least 90% of the amino acid residues of the fragment are identical to corresponding residues of the amino acid sequence as shown in SEQ ID NO:1, preferably at least 92%, most preferably at least 95%. Percent sequence identity is determined by conventional methods. The degree of identity of the amino acid sequence of the fragment to SEQ ID NO:1 (or of any other native fish parvalbumin sequence) may be determined by comparing the amino acid sequence of the fragment and the reference sequence, such as SEQ ID NO:1 using, for example, the program “Blast 2 sequences” (Tatusova et al. (1999) FEMS Microbiol. Lett. 174, 247-250). The parameters which are used in this context are: matrix: BLOSUM 62; gap open: 11; gap extension: 1; X drop off: 50; expect: 10; word size: 3; filter: no.

In another embodiment, the polypeptide of the invention comprises a biologically active fragment of the amino acid sequence as shown in SEQ ID NO:1 or one of the other sequences of FIG. 7, with a length of at least 15 amino acids. The fragment consists of at least 15 consecutive amino acids of the amino acid sequence as shown in FIG. 7, preferably at least 20 consecutive amino acids, more preferably at least 25 consecutive amino acids, most preferably at least 30 consecutive amino acids.

The polypeptides of the invention may essentially consist of a biologically active fragment of the polypeptides described above. This fragment essentially consists of at least 10 consecutive amino acids of the polypeptide described above (a), preferably at least 15 consecutive amino acids, more preferably at least 20 consecutive amino acids, even more preferably at least 25 consecutive amino acids, most preferably at least 30 consecutive amino acids. The amino acid sequence of the fragment is at least 80% identical to corresponding residues of the amino acid sequence as shown in SEQ ID NO:1 or in another wild type sequence set forth in FIG. 7. The degree of amino acid sequence identity is determined as described, supra. The sequence identity of the fragments to the amino acid sequence of SEQ ID NO:1 or other wild type sequence of FIG. 7, is preferably at least 85%, more preferably at least 90%, most preferably at least 95%.

In another embodiment, the polypeptide of the invention consists of a biologically active fragment of an amino acid sequence in FIG. 7. This fragment consists of at least 10 consecutive amino acids of the amino acid sequence, preferably at least 15 consecutive amino acids, more preferably at least 20 consecutive amino acids, even more preferably 25 consecutive amino acids, most preferably at least 30 consecutive amino acids. Preferred polypeptides comprise at least the amino acids forming one of the EF hand motifs (amino acids D52-E63 or D91-E102 of the amino acid sequence as shown in SEQ ID NO:1). Examples are the polypeptides consisting substantially of the amino acid sequence as shown in SEQ ID NO:5 (first EF hand motif) or SEQ ID NO:6 (second EF hand motif), respectively, optionally coupled to a carrier molecule such as keyhole limpet hemocyanin (KLH). See also SEQ ID NO: 2 and 3.

By the term “biologically active” is meant that the polypeptide induces an allergic response in a mammal. The polypeptides of the present invention, usually have reduced allergenic activity compared to wild type fish parvalbumin, particularly with regard to Cyp c 1.01. According to the invention the term “allergenic activity” denotes the capability of a compound or composition to induce an allergic reaction in a sensitized mammal, e.g. in a fish allergic patient. An allergic reaction may be mast cell degranulation, positive skin reaction and/or nasal reaction. The allergenic activity is preferably defined in suitable in vitro or in vivo tests. The allergenic activity may be determined in a skin test as described in van Hage-Hamsten et al. J. Allergy Clin. Immunol. 1999, 104, pp. 969-977 or in Pauli et al. Clin. Exp. Allergy 2000, 30, pp. 1076-1084. The allergenic activity of wild type Cyp c 1.01 may be determined using recombinantly produced Cyp c 1.01 essentially consisting of the amino acid sequence as shown in SEQ ID NO:1.

Preferably the allergenic activity of the polypeptide is less than 50% of the allergenic activity of the wild type fish parvalbumin, particularly Cyp c 1.01. More preferably the allergenic activity of the polypeptide is less than 25% of the wild type protein. In the most preferred embodiment the polypeptide has substantially no allergenic activity. Generally, the histamine release induced by the polypeptide of the invention is significantly reduced compared to the histamine release induced by Cyp c 1.01. A preferred in vitro test for determining the histamine release is the basophil histamine release assay as described in Vrtala et al., J. Clin. Invest. 1997, 99, pp. 1673-1681. Preferably, the histamine release is reduced by at least 25%, more preferably by at least 50%, most preferably by at least 75%, determined at that concentration of allergen at which Cyp c 1.01 shows maximum histamine release.

The allergenic activity referred to above may vary depending on the serum or patient examined. The values given above refer to the average allergenic activity, e.g. determined through examination of at least 20 randomly selected fish allergic patients or their sera.

The polypeptides of the invention usually show reduced binding to IgE antibodies from fish allergic patients compared with wild type fish parvalbumin, such as Cyp c 1.01 (SEQ ID NO:1). The IgE binding activity is preferably reduced by at least 25%, more preferably by at least 50%, most preferably by at least 75%. In one embodiment, recombinant Cyp c 1.01 essentially consisting of the amino acid sequence as shown in SEQ ID NO:1 can be used to determine the IgE binding activity of wild type Cyp c 1.01. IgE binding of polypeptides may be determined in an ELISA or by Western blot analysis or dot blot experiments using serum from a fish allergic patient. Fish allergy is diagnosed according to a case history indicative for fish allergy, positive skin test reaction to fish allergens and/or the detection of specific IgE antibodies to fish allergens in serum. Dot blots can be quantified by measuring the amount of ¹²⁵I-labeled anti-human IgE antibodies by gamma counting as described (Niederberger et al. J. Allergy Clin. Immunol. 1998, 102, 579-591).

The IgE binding activity referred to above may vary depending on the serum or patient examined. The values given above refer to the average IgE binding activity, e.g. determined through examination of at least 20 randomly selected fish allergic patients or their sera (see Table 3).

In some cases, a given polypeptide of the invention may show reduced binding activity to IgE antibodies of some fish allergic patients, whereas it shows unaltered or even increased binding to IgE antibodies of other fish allergic patients. Such polypeptides are also within the scope of the invention, since they are useful in the treatment of patients from which the IgE antibodies with reduced binding were obtained.

The invention further relates to a method for the treatment of a fish allergic patient, comprising (a) determining the binding activity of a polypeptide of the invention to IgE antibodies from the serum of said patient; (b) determining the binding activity of Cyp c 1.01 to the said IgE antibodies; and (c) treating the patient with the polypeptide of the invention if the IgE binding activity determined in (a) is significantly lower than the IgE binding activity determined in (b). Preferably, the IgE binding activity determined in (a) is less than 75%, preferably less than 50%, more preferably less than 25%, most preferably less than 10% of that determined in (b).

The invention further relates to a method for making a medicament for the treatment of a fish allergic patient. In one embodiment, the method first comprises preparing fish parvalbumin mutants, as described below. The method further comprises (a) determining in vitro the binding activity of a polypeptide of the invention to IgE antibodies from the serum of said patient; (b) determining in vitro the binding activity of Cyp c 1.01 to the said IgE antibodies; and (c) selecting the polypeptide of the invention and mixing it with a pharmaceutically acceptable carrier or diluent if the IgE binding activity determined in (a) is significantly lower than the IgE binding activity determined in (b). Preferably, the IgE binding activity determined in (a) is less than 75%, preferably less than 50%, more preferably less than 25%, most preferably less than 10% of that determined in (b).

Preferably, the fish parvalbumin-derived polypeptides of the invention induce IgG antibody responses in vivo. Therefore, the polypeptides described above generally comprise at least one IgG epitope. A polypeptide comprises at least one IgG epitope when it is capable of eliciting an IgG antibody response in an individual or a test animal. A corresponding test for determining an IgG response is described in European Patent Application No. 02021837 relating to hypoallergenic polypeptides based on pollen allergens. More preferably, these IgG antibodies are “blocking antibodies” or “protective antibodies” which prevent IgE antibodies from binding to Cyp c 1.01. A significant reduction of allergic symptoms may be achieved in this way.

The invention further relates to a hybrid polypeptide comprising more than one biologically active fragment of a mutated fish parvalbumin. In one embodiment, the hybrid polypeptide essentially consists of 2 or 3 fragments of different fish parvalbumins and/or of different polypeptides described above and in Example 7 and FIG. 7. The hybrid polypeptide preferably has a length of at least 90 amino acids. In another embodiment, the invention relates to fusion proteins comprising one or more polypeptides of the invention.

It has been found that the polypeptides of the invention which carry mutations corresponding to the mutations represented by amino acid sequences SEQ ID NO:2, SEQ ID NO:3, SEQ ID NO:4, SEQ ID NO:5 or SEQ ID NO:6 have unexpected advantageous properties. The amino acid positions targeted in these amino acid sequences can be substituted or deleted in other fish parvalbumins as well, such as those set forth in Table 1 and in FIG. 7. Therefore, the present invention relates to a mutated polypeptide derived from a fish parvalbumin, wherein in respect to the wild type sequence of the fish parvalbumin amino acid positions have been substituted or deleted which correspond to the amino acid residues which are substituted or deleted in respect to SEQ ID NO:1 in SEQ ID NO:2, SEQ ID NO:3, SEQ ID NO:4, SEQ ID NO:5 or SEQ ID NO:6. The identification of amino acid positions in other fish parvalbumins which correspond to the amino acid positions mutated in any one of SEQ ID NO:2 to 6 is within the level of ordinary skill. For example, the skilled person may align anyone of the sequences as shown in FIG. 7, such as SEQ ID NO:1 to 6, with the amino acid sequence of a given fish parvalbumin using the program “Blast 2 sequences” described above. From the alignment the corresponding amino acid positions to be mutated can easily be derived. As an example, FIG. 6 shows an alignment of Cyp c 1.01 parvalbumin and parvalbumin from Ictalurus punctatus (SEQ ID NO:10). The amino acid positions to be substituted or deleted correspond to D52, D54, D91 and/or D93 of SEQ ID NO:1. These positions correspond to D68, D70, D107 and D109 of the amino acid sequence of parvalbumin from Ictalurus punctatus (SEQ ID NO:10). FIG. 7 presents sequence alignments from a variety of fish parvalbumins. The polypeptides derived from fish parvalbumins may be comprised in a larger polypeptide or be coupled to a suitable carrier protein such as KLH. They may also have reduced allergenic activity and IgE binding capacity compared with their respective wild type forms and are capable of inducing an IgG response as described, supra. The amino acid sequences of several fish parvalbumins are known:

TABLE 1
Examples of naturally occurring and mutated fish parvalbumins.

| | accession | | SEQ |
|---|---|---|---|
| Definition | No. | organism | ID NO: |
| Cyp c WT | | Cyprinus carpio | 1 |
| Cyp c Mut D52A, D54A | | artificial | 2 |
| Cyp c Mut D91A, D93A | | artificial | 3 |
| Cyp c Mut D52A, D54A | | artificial | 4 |
| D91A, D93A | | | |
| Cypc WT fragment | | | 5 |
| D52-63 | | | |
| Cypc WT fragment | | | 6 |
| D91-102 | | | |
| parvalbumin isoform 1b | AAO33403 | Danio rerio | 7 |
| Parvalbumin alpha (A1) | P09227 | Cyprinus carpio | 8 |
| Cyp c 1.02 | CAC83659 | Cyprinus carpio | 9 |
| parvalbumin | AAO25757 | Ictalurus punctatus | 10 |
| Gad m 1 | Q90YK9 | Gadus morhua | 11 |
| parvalbumin | NP_571591 | Danio rerio | 12 |
| parvalbumin isoform 1c | AAO33402 | Danio rerio | 13 |
| parvalbumin I | 1206380A | Electrophorus sp. | 14 |
| Sco j 1 | P59747 | Scomber japonicus | 15 |
| Primer mut CD | | artificial | 16 |
| Primer mut EF | | artificial | 17 |
| DNA encoding | | Cyprinus carpio | 18 |
| SEQ ID NO: 1 | | | |
| Clu h 1 | FM178220 | herring | 19 |
| Thu a 1 | FM178217.1 | Tuna | 20 |
| Gad c 1 | AY035584 | Cod fish | 21 |
| Sal s 1 | Q91482.1 | Atlantic salmon | 22 |
| Clu h 1mut D52A D54A | | artificial | 23 |
| Thu a 1 mut D52A D54A | | artificial | 24 |
| Gad c 1 Mut D52A, D54A | | artificial | 25 |
| Sco j 1 Mut D52A, D54A | | artificial | 26 |
| Sal s 1 Mut D52A, D54A | | artificial | 27 |
| Clu h 1 Mut D91A, D93A | | artificial | 28 |
| Thu a 1 Mut D91A, D93A | | artificial | 29 |
| Gad c Mut D91A, D93A | | artificial | 30 |
| Sco j 1 Mut D91A, D93A | | artificial | 31 |
| Sal s 1 Mut D91A, D93A | | artificial | 32 |
| Clu h 1 Mut | | artificial | 33 |
| D52A, D54A, D91A, | | | |
| D93A Thu a 1 | | artificial | 34 |
| MutD52A, D54A, D91A, | | | |
| D93A Gad c 1 | | artificial | 35 |
| MutD52A, D54A, D91A, | | | |
| D93A Sco j 1 | | artificial | 36 |
| MutD52A, D54A, D91A, | | | |
| D93A Sal s 1 | | artificial | 37 |
| MutD52A, D54A, D91A, | | | |
| D93A | | | |

Polypeptides derived from fish parvalbumins other than Cyp c 1.01 preferably comprise substitutions or deletions at amino acid positions corresponding to those substituted in any one of SEQ ID NO:2 to 6. See e.g. SEQ ID NO: 23-37 in Table 1.

The invention pertains to a non-naturally occurring polypeptide derived from a wild type fish parvalbumin, comprising an amino acid sequence in which in respect to the amino acid of said wild type fish parvalbumin the amino acids at the positions corresponding to positions 52 and 54 of SEQ ID NO:1 are substituted. See Table 1 and FIG. 7.

The invention further relates to a non-naturally occurring polypeptide derived from a wild type fish parvalbumin, comprising an amino acid sequence in which in respect to the amino acid of said wild type fish parvalbumin the amino acids at the positions corresponding to positions 91 and 93 of SEQ ID NO:1 are substituted. See Table 1 and FIG. 7.

The invention further relates to a non-naturally occurring polypeptide derived from a wild type fish parvalbumin, comprising an amino acid sequence in which in respect to the amino acid of said wild type fish parvalbumin at least two of the amino acids at the positions corresponding to positions 52, 54, 91 and 93 of SEQ ID NO:1 are substituted. See Table 1 and FIG. 7.

An example is a non-naturally occurring polypeptide derived from Danio rerio parvalbumin isoform 1b, comprising an amino acid sequence in which in respect to the amino acid sequence as shown in SEQ ID NO:7 the amino acids at positions 52 and 54; or at positions 91 and 93; or preferably at positions 52, 54, 91 and 93 are substituted. Another example is a non-naturally occurring polypeptide derived from Cyprinus carpio parvalbumin alpha (A1), comprising an amino add sequence in which in respect to the amino acid sequence as shown in SEQ ID NO:8 the amino acids at positions 51 and 53; or at positions 90 and 92; or preferably at positions 51, 53, 90 and 92 are substituted.

Thus, in one embodiment, the invention relates to a method of preparing polypeptides that are useful in the treatment and prophylaxis of allergic reactions against fish parvalbumin, the method comprising mutating wild type fish parvalbumin in at least one amino acid residue that corresponds to amino acid residues 52, 54, 91 and 93 of SEQ ID NO:1, when said wild type fish parvalbumin amino acid sequence is aligned with SEQ ID NO:1, as described above. Preferably, the mutation comprises substituting at least one of amino acid residues 52, 53, 91 or 93 for a non-native amino acid. The allergenic activity of such mutated polypeptide can then be compared to that of SEQ ID NO:1 with those mutated polypeptides having reduced allergenic activity being selected for use in treatment and prophylaxis and related methods of making medicaments for use in treatment and prophylaxis. Methods of assessing allergenic activity are described above.

Wild type Cyp c 1.01 represented by the amino acid sequence as shown in SEQ ID NO:1 and proteins essentially consisting of wild type Cyp c 1.01 are not polypeptides of the present invention. Naturally occurring fish parvalbumins or recombinant proteins consisting of the same amino acids as the naturally occurring fish parvalbumins are not polypeptides of this application.

Hypoallergenic parvalbumins do not necessarily consist only of amino acid sequences derived from parvalbumin proteins. It is possible that they also comprise ‘tag’ sequences which will facilitate the purification of the proteins after expression in the host cells. An example for such a ‘tag’ is the hexahistidine tag (SEQ ID No: 38) which allows purification of the protein by Ni²⁺ chelate chromatography. However, a number of other tags is also known and used in the art.

The present invention also provides polynucleotide molecules, including DNA and RNA molecules, that encode the polypeptides disclosed herein. Those skilled in the art will readily recognize that, in view of the degeneracy of the genetic code, considerable sequence variation is possible among these polynucleotide molecules. The polynucleotide may be single or double-stranded. It is to be recognized that according to the present invention, when a polynucleotide is claimed as described herein, it is understood that what is claimed are both the sense strand, the antisense strand and the DNA as double-stranded having both the sense and antisense strand annealed together by their respective hydrogen bonds. Also claimed is the messenger RNA (mRNA) which encodes the polypeptides of the present invention. Messenger RNA will encode a polypeptide using the same codons as those used by DNA, with the exception that each thymine nucleotide (T) is replaced by a uracil nucleotide (U).

Methods for preparing DNA and RNA are well known in the art. A full-length clone encoding fish parvalbumin such as Cyp c 1.01 can be obtained by conventional cloning procedures. The DNA encoding such fish parvalbumin may be amplified by polymerase chain reaction (PCR) employing suitable specific primers. The polynucleotides of the present invention may also be synthesized chemically, for example using the phosphoramidite method. The coding sequence of Cyp c 1.01 cDNA is shown in SEQ ID NO:18.

The invention further relates to a vector or plasmid containing a polynucleotide as described above. In general, the polynucleotide sequence encoding a polypeptide of the invention is operably linked to other genetic elements required for its expression, generally including a transcription promoter and terminator, within an expression vector. The vector will also commonly contain one or more selectable markers (e.g., genes coding for antiviotics resistance) or one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

Another aspect of the invention is a host cell transformed or transfected with a vector or a plasmid according to the invention. The host cells may be prokaryotic or eukaryotic cells. Prokaryotic host cells, including strains of the bacteria Escherichia coli, Bacillus and other genera are useful host cells within the present invention. Yeast, insect cells or mammalian cell lines like CHO cells can be used as eukaryotic host cells. Techniques for transforming these hosts and expressing foreign DNA sequences cloned therein are well known in the art (see, e.g., Sambrook et al., Molecular Cloning, a Laboratory Manual, Cold Spring Harbor Laboratory, 1989).

The host cells of the invention may be used to produce the polypeptides of the invention. Yet another aspect of the invention therefore is a method of preparing a polypeptide according to the invention comprising culturing host cells described above under conditions that said polypeptide is expressed and optionally recovering said polypeptide from the host cells. When expressing a polypeptide of the invention in bacteria such as E. coli, the polypeptide may be retained in the cytoplasm, possibly as insoluble inclusion bodies. In this case, the cells are lysed and the inclusion bodies are recovered and denatured using, for example, guanidine isothiocyanate or urea. The denatured polypeptide can then be refolded by diluting the denaturant, such as by dialysis against a solution of urea and a combination of reduced and oxidized glutathione, followed by dialysis against a buffered saline solution.

Transformed or transfected host cells are cultured according to conventional procedures in culture medium containing nutrients and other components required for the growth of the chosen host cells. A variety of suitable media, including defined media and complex media, are known in the art and generally include a carbon source, a nitrogen source and minerals. It is preferred to purify the peptides of the present invention to ≧80% purity, more preferably ≧95% purity, and particularly preferred is a pharmaceutically pure state that is greater than 99.9% pure with respect to contaminating macromolecules, particularly other proteins and nucleic acids, and free of infectious and pyrogenic agents. Preferably, a purified polypeptide of the invention is substantially free of other polypeptides. Expressed recombinant polypeptide of the invention can be purified using fractionation and/or conventional purification methods and media. Ammonium sulfate precipitation and acid or chaotrop extraction may be used for fractionation of samples. The polypeptides of the invention may also be isolated by affinity chromatography using antibodies directed to the polypeptide. Shorter polypeptides are preferably purified using HPLC. Methods of protein purification are described e.g. in Methods in Enzymology, Volume 182. Guide to Protein Purification. Academic Press New York 1990 and Scopes, Protein Purification. Springer Verlag, Heidelberg 1994.

The polypeptides of the invention may also be prepared through chemical synthesis, for example, as described by Merryfield, J. Am. Chem. Soc. 85:2149, 1963 and Etherton et al., Solid Phase Peptide Synthesis: A Practical Approach, IRL Press, Oxford 1989.

The invention further relates to the use of the polypeptide or polynucleotide or cell of the invention for the manufacture of a medicament for treating and/or preventing an allergic disorder. The disorder usually is an allergy to one or more fish allergens, e.g. to Cyp c 1.01. Preferably, the disorder is IgE-mediated fish hypersensitivity. It has been found that Cyp c 1.01 contains most of the relevant IgE epitopes of different fish allergens. Therefore, the polypeptides or polynucleotides may be used in the treatment of almost any fish allergy. Preferably, the allergic disorder to be treated is allergy to Cyp c 1.01 or to at least one of the proteins listed in table 1 above. The medicament may be used for the therapeutic treatment of an allergic disorder or for prophylactic vaccination to prevent development of the disorder.

The invention also pertains to a pharmaceutical composition comprising at least one polypeptide, variant or biologically active fragment or at least one polynucleotide of the invention. The composition may further comprise a pharmaceutically acceptable carrier or diluent. Preferably, the polypeptide of the invention has been coupled to a carrier molecule such as KLH.

Another aspect of the invention is a pharmaceutical kit comprising at least one polypeptide or polynucleotide of the invention. The kit may comprise two or more different polypeptides or two or more different polynucleotides according to the present invention. In one embodiment the kit comprises at least one mutated polypeptide derived from Cyp c 1.01 and at least one mutated polypeptide derived from another fish allergen. Other allergens from carp or from other fish and their epitopes may be contained.

For pharmaceutical use, the polypeptides, variants and fragments of the present invention are formulated for oral or parenteral, particularly subcutaneous, delivery according to conventional methods. In general, pharmaceutical formulations will include a polypeptide of the invention in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, Ed., Mack Publishing Co., Easton, Pa., 19th Edition 1995. Therapeutic or prophylactic doses will generally be in the range of 0.1-100 μg per injection in a volume of 100-200 μl, with the exact dose determined by the physician according to accepted standards, taking into account the nature and severity of the condition to be treated, patient traits, etc. Determination of dose is within the level of ordinary skill in the art. The amount may vary depending on the mode of treatment. During immunotherapy treatment single doses of about 25 μg to 75 μg can be administered in a volume of about 100 μl per injection. In case of oral administration a dosage of 0.1 μg to 50 mg can be envisaged. In the case of vaccinations, patients are usually not treated several times a day except for “rush immunotherapy”. Common immunotherapies include about 8 vaccinations that are administered in intervals of one to two weeks and that are continued over a period of 2 to 3 years. Preferably, 4 injections per year with an interval of 3 months over 3 to 5 years are applied. In a particular embodiment, more than one polypeptide is contained in the pharmaceutical composition.

The polynucleotide of the invention is useful in DNA vaccination to treat and/or prevent an allergic disorder, preferably fish allergy.

The invention also relates to the use of hypoallergenic parvalbumin polypeptides, polynucleotides encoding hypoallergenic polypeptides or cells expressing hypoallergenic parvalbumins for the preparation of a medicament for the treatment of IgE-mediated fish hypersensitivity. Such a medicament may be composed of the polynucleotide, which could be directly used for DNA-based vaccination against fish allergy. Alternatively, the hypoallergenic polypeptide might be used to prepare formulations for oral, sublingual or parenteral treatment of Type I allergic disorders. Modes of application for parenteral treatments may be nasal administration of the hypoallergenic parvalbumin or subcutaneous injection of adjuvant-bound hypoallergenic polypeptides. Furthermore, possible applications comprise also cell-based forms of immunotherapy. In this case antigen presenting cells are transformed with vectors containing the polynucleotide sequence of the hypoallergen and they then express the hypoallergen in vivo.

The invention also relates to the pharmaceutical composition of the medicament containing a polypeptide, polynucleotide or a cell according to the invention. This includes pharmaceutically acceptable carriers or diluents like buffers or salt solutions. In a particular embodiment the pharmaceutical composition also contains an adjuvant.

One or both of the functional calcium-binding sites of rCyp c 1.01 were modified by replacement of two acidic amino acids by non-polar residues. Surprisingly, introduction of such point mutations in only one of the calcium-binding domains displayed already severe effects on the IgE reactivity of the allergen. However, variable changes of IgE-binding capacity were observed, depending on the sera used and some patients even exhibited higher IgE reactivities to such parvalbumin mutants than to the wild-type protein. In contrast, modifications in both of the calcium-binding domains apparently resulted in disruption of all the important IgE epitopes, since IgE reactivity was completely abolished in all sera tested. With a few amino acid changes the inventors thus succeeded to convert a highly reactive food allergen (rCyp c 1.01) into hypoallergenic molecules which can be used for allergen-specific immunotherapy of fish allergy.

In view of the stability of parvalbumin, it could not have been expected that only few point mutations (amino acid substitutions) would be sufficient to produce a polypeptide exhibiting reduced allergenic activity.

The remarkable IgE cross-reactivity among parvalbumins of commonly consumed fish species and the fact that recombinant carp parvalbumin (rCyp c 1.01) contains most of the IgE epitopes present in allergen extracts of several fish species (Swoboda et al., 2002) suggests that rCyp c 1.01-based hypoallergenic molecules can be used to treat the majority of fish allergic patients.

Hypoallergenic parvalbumin molecules will therefore open new avenues for immunotherapy of IgE-mediated fish hypersensitivity. Since the hypoallergenic variants consist of defined components, it will be possible to produce well defined formulations for vaccination treatment. Furthermore, the lack of allergenic activity will allow administration of the hypoallergenic parvalbumins at high doses, comparable to vaccines regularly used for prevention of viral infections, which will result in a high efficacy of the treatment. In this respect it may be considered to use these molecules also for prophylactic vaccination or tolerance induction in not yet sensitised individuals.

The various embodiments of the invention described herein may be cross-combined, in particular the preferred forms of the aspects of the invention.

### DESCRIPTION OF DRAWINGS

FIG. 1: Far-ultraviolet circular dichroism analysis of purified recombinant parvalbumin (Wild-type) and the parvalbumin mutants (Mut-EF, Mut-CD, Mut-CD/EF). Results are expressed as the mean residue ellipticity (Θ) (y-axis) at a given wave length (x-axis).

FIG. 2: SDS-PAGE and immunoblot analysis of purified recombinant parvalbumin (Wild-type) and of the parvalbumin mutants (Mut-EF, Mut-CD, Mut-CD/EF). A, Coomassie brilliant blue-stained SDS-PAGE. B, Immunoblot showing IgE reactivity to the serum of a fish allergic patient. Molecular weights are indicated at the left margins.

FIG. 3: IgE reactivity of four fish allergic patients to nitrocellulose-dotted duplicates of recombinant parvalbumin (Wild-type) and of the parvalbumin mutants (Mut-EF, Mut-CD, Mut-CD/EF).

FIG. 4: Inhibition of IgE binding to nitrocellulose-blotted natural carp parvalbumin after preincubation of the serum from a fish allergic patient with recombinant parvalbumin (Wild-type), with the parvalbumin mutants (Mut-EF, Mut-CD, Mut-CD/EF) or with bovine serum albumin (lane BSA). Molecular weights are indicated at the left margin.

FIG. 5: Biological activity of purified recombinant parvalbumin (Wild-type) and of the parvalbumin mutants (Mut-EF, Mut-CD, MutCD/EF). Induction of histamine release from basophils of a fish allergic patient stimulated by various concentrations (μg/ml) of the recombinant proteins (x-axis). The percentage of histamine released into the supernatant is displayed on the y-axis.

FIG. 6: Alignment of Cyp c 1.01 (SEQ ID NO:1) with amino acid residues 17 to 125 of Ictalurus punctatus. (SEQ ID NO: 10). The amino acid residues in the line between the two aligned sequences show where the two sequences are the same.

FIG. 7: Comparison of the protein sequences (amino acid residues 1-109) of the wildtype (WT) and mutated (Mut) parvalbumins from carp (Cypc), herring (Cluh), tuna (Thua), codfish (Gadc), chub mackerel (Scoj) and Atlantic salmon (Sals). Exchanged amino acids are indicated by underlining in the sequences of the mutants.

FIG. 8: The parvalbumin mutants show no IgE reactivity. Nitrocellulose-blotted extracts of induced E. coli cultures expressing wildtype and mutated (Mut) parvalbumins from carp (Cyp c 1), Atlantic salmon (Sal s 1), tuna (Thu a 1), chub mackerel (Sco j 1), herring (Clu h 1) and codfish (Gad c 1) were exposed to serum IgE from four fish allergic patients. The molecular masses are indicated in the left margins.

The invention is further illustrated by the following non-limiting examples:

### Example 1

### Quantitative IgE Inhibition Studies Show that Recombinant Carp Parvalbumin Contains the Majority of IgE Epitopes Present in Various Fish Species

To investigate and quantify the cross-reactive potential of recombinant carp parvalbumin, sera from 16 fish-allergic patients were preincubated with recombinant wild-type parvalbumin, expressed and purified as previously described (Swoboda et al., 2002), and then exposed to allergen extracts from cod, tuna, and salmon. Quantification of remaining IgE reactivity by CAP-FEIA measurements revealed a reduction of IgE binding to cod extract ranging between 62% and 96%, to tuna extract between 33% and 98% and to salmon extract between 41% and 95% (Table 2). These findings indicated that recombinant carp parvalbumin represents a highly cross-reactive allergen, which contains a large portion of IgE epitopes present in allergen extracts of various fish species.

Experimental Protocol:

Sera from 16 fish-allergic patients were preincubated with 5 μg recombinant carp parvalbumin or, for control purposes, with 5 μg BSA. Remaining IgE reactivity to cod, tuna and salmon fish extracts was quantified using the CAP-FEIA system (Pharmacia, Uppsala, Sweden). The percentage inhibition of IgE binding to fish extracts after preabsorption to the recombinant allergen was calculated as ((cpm_{BSA}−cpmₚₐᵣᵥ)/cpm_{BSA})×100, where cpm_{BSA} and cpmₚₐᵣᵥ indicate IgE binding after preabsorption with BSA and recombinant carp parvalbumin, respectively.

TABLE 2
Percentage inhibition of IgE reactivity to cod, tuna and salmon
protein extracts after preabsorption of sera with recombinant carp
parvalbumin (as measured in the CAP-FEIA system).

| Serum | Cod | Tuna | Salmon |
|---|---|---|---|
| 1 | 62% | 74% | 41% |
| 2 | 87% | 95% | 88% |
| 3 | 96% | 45% | 83% |
| 4 | 83% | 62% | 74% |
| 5 | 64% | 45% | 69% |
| 6 | 90% | 81% | 73% |
| 7 | 69% | 43% | 58% |
| 8 | 66% | 33% | 50% |
| 9 | 85% | 62% | 59% |
| 10 | 85% | 85% | 62% |
| 11 | 85% | 83% | 70% |
| 12 | 82% | 95% | 85% |
| 13 | 91% | 96% | 92% |
| 14 | 95% | 98% | 91% |
| 15 | 88% | 96% | 94% |
| 16 | 68% | 80% | 95% |
| Mean | 76% | 69% | 70% |

### Example 2

### Construction of Parvalbumin Mutants

In order to modify the carp parvalbumin cDNA Cyp c 1.01 (EMBL accession number AJ292211) in one or both of the functional calcium binding sites (CD- or EF domain), site-directed mutagenesis was carried out using the Chameleon Double-Stranded, Site-Directed Mutagenesis Kit (Stratagene, La Jolla, Calif.). The Ca²⁺-binding domains were mutated by replacing the first and third amino acid of the calcium binding loops (Asp) by non-polar Ala residues. Mutagenesis experiments were performed according to the manufacturer's instructions with two synthetic oligonucleotides (mutCD and mutEF) using Cyp c 1.01 DNA cloned into the expression vector pET-17b (Swoboda et al., 2002) as a template. Both oligonucleotides encompassed 45 bp of the parvalbumin cDNA. Primer mutCD (5′-AAG GCC TTT GCT GTC ATT GCC CAA GCC AAG AGC GGC TTC ATT GAG-3′; SEQ ID NO:16) introduced changes in codons 52 (GAC→GCC) and 54 (GAC→GCC) and mutEF (5′-GCC TTC CTG AAA GCT GGA GCC TCT GCT GGT GAT GGC MG ATT GGA-3′; SEQ ID NO:17) in codons 91 (GAC→GCC) and 93 (GAT→GCT), respectively. Modified codons are underlined in the oligonucleotide sequences. Modifications were confirmed by dideoxynucleotide chain-termination sequencing (Sanger et al., 1977) using a T7 sequencing kit (Pharmacia, Uppsala, Sweden) and resulting proteins were termed Mut-CD (mutation in CD domain), Mut-EF (mutation in EF domain) and Mut-CD/EF (mutated in both domains).

### Example 3

### Expression and Purification of Recombinant Carp Parvalbumin and of the Parvalbumin Mutants

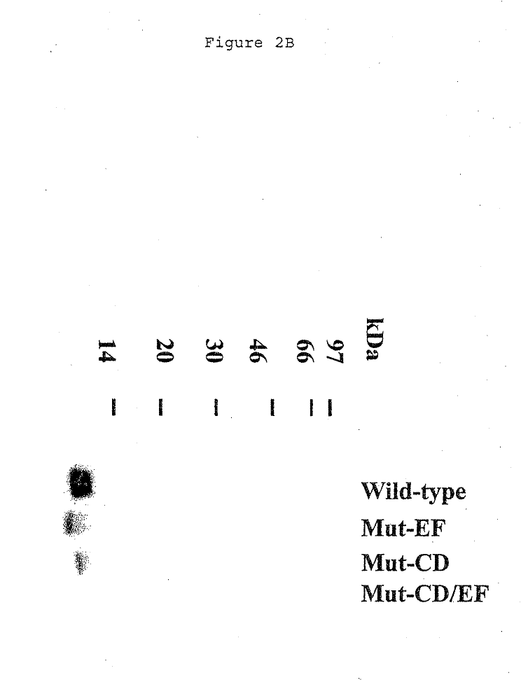

Recombinant wild-type parvalbumin and the three parvalbumin mutants were expressed in Escherichia coli strain BL21(DE3). Isopropylthiogalactopyranoside (IPTG)-induced expression of Mut-CD and Mut-EF proteins resulted in a protein production similar to that observed for the wild-type protein (Swoboda et al., 2002), with 25-30% recombinant protein per total E. coli protein. However, bacteria expressing Mut-CD/EF grew more slowly and only approximately 2-3% of total bacterial protein represented Mut-CD/EF protein. Recombinant proteins were purified from the soluble cytoplasmic fractions of bacterial extracts by anion exchange chromatography to about 95% homogeneity as judged by Coomassie brilliant blue staining of 15% sodium dodecyl sulfate polyacrylamid gels (SDS-PAGE; FIG. 2A) (Laemmli 1970).

Experimental Protocol:

Expression of recombinant proteins was induced in E. coil BL21(DE3) cells, which had been grown in LB-medium containing 100 mg/I ampicillin to an OD₆₀₀ of 0.4. After an induction of two hours at 37° C. the majority of the protein was found in the soluble fractions of the bacterial extract. Cells were harvested, resuspended in 10 mM Tris (pH 7.5), 1 mM PMSF and lysed by several freeze-thaw cycles with freezing in liquid nitrogen and thawing at 10°-15° C. In case of Mut-CD/EF thawed cells were finally also mechanically disrupted with an ultraturrax (Polytron, Kin ematica AG, Switzerland). After centrifugation at 20,000×g for 30 min at 4° C. the cleared supernatant was applied to a DEAE cellulose-Sepharose column (DEAE Sepharose Fast Flow, Pharmacia Biotech, Uppsala, Sweden). Fractions containing the purified proteins were eluted with a linear salt gradient (0-0.5 M NaCl in 10 mM Tris, pH7.5) and dialysed against PBS (pH 7.4).

### Example 4

### Structural Analysis of the Parvalbumin Mutants Shows that Point-Mutations Lead to Significant Conformational Changes

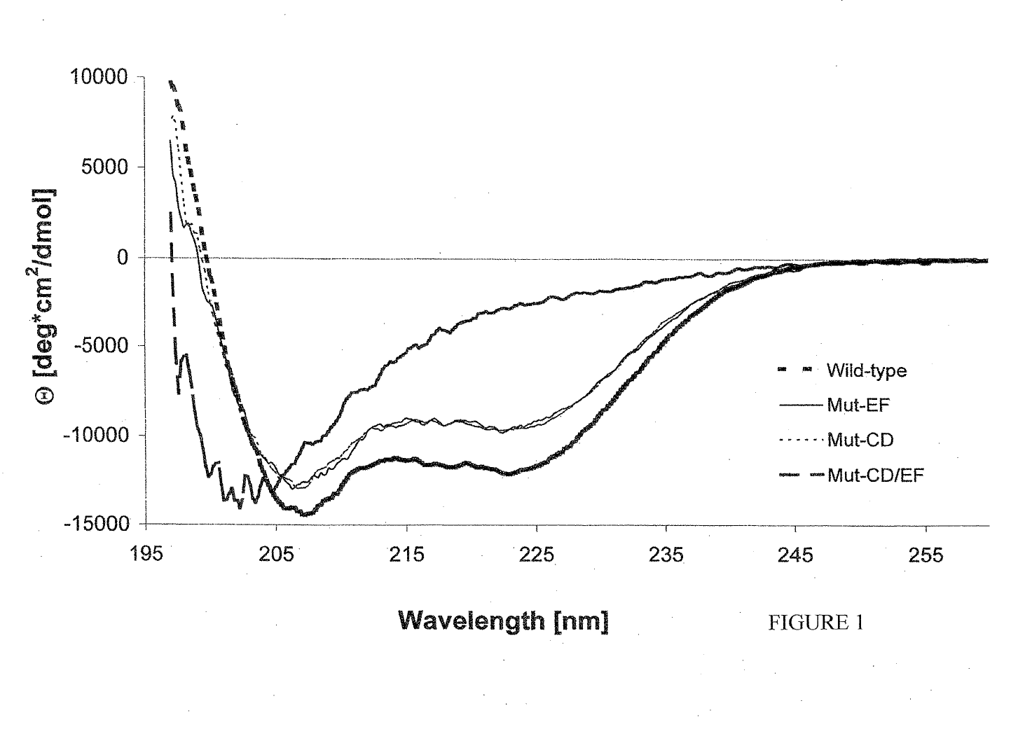

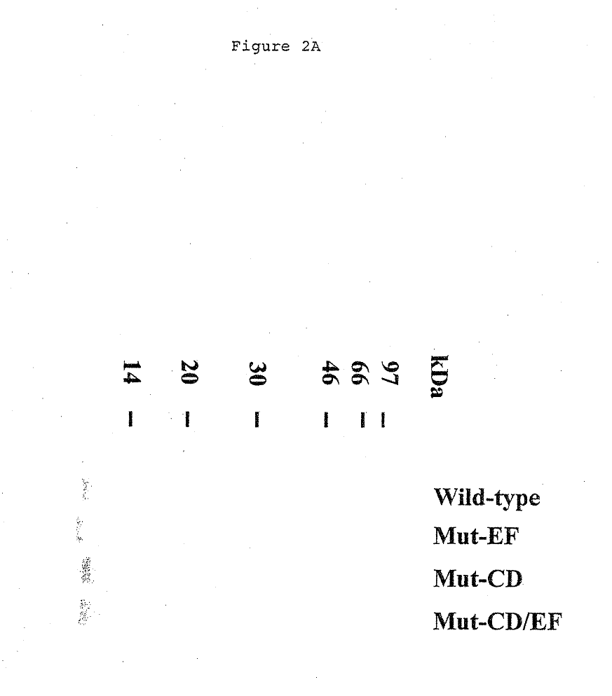

The far-ultraviolet CD spectrum (FIG. 1) of the recombinant wild-type parvalbumin was characterised by two broad minima at 208 nm and 223 nm and a strong maximum below 200 nm. Such a shape is typical for a well-structured protein with a considerable amount of α-helices. Overall the spectra of the mutants that had been modified in one of the calcium binding domains (Mut-CD and Mut EF) showed the same characteristics of folded proteins as the wild-type protein. However, mutations introduced in both calcium binding domains (as in Mut-CD/EF) led to remarkable differences in the shape of the far-UV spectrum. The observed single broad minimum at about 202 nm indicates a significant decrease in the α-helical content and in the secondary structure of the molecule and a transition into a random coiled, unfolded conformation. By introduction of a few targeted point mutations the inventors had thus succeeded to significantly modify the structure of this highly stable allergen.

Experimental Protocol:

Circular dichroism (CD) measurements were performed on a Jasco (Tokyo, Japan) J-715 spectropolarimeter with protein concentrations between 12.3-24.0 μM using a 1 mm path-length quartz cuvette (Helima, Mullheim, Baden, Germany) equilibrated at 20° C. Spectra were recorded with 0.2 nm resolution at a scan speed of 50 nm/min and resulted from averaging of 3 scans. The final spectra were corrected by subtracting the corresponding baseline spectrum obtained under identical conditions. Results are expressed as the mean residue ellipticity (Θ) at a given wavelength.

### Example 5

### Parvalbumin Mutants Show Reduced IgE Binding Capacity

The IgE reactivity to recombinant wild-type and mutated parvalbumins was analysed in immunoblot and dot blot experiments. The immunoblot shown in FIG. 2B was probed with the serum of a fish allergic patient and exemplifies the IgE binding capacity of the denatured, immobilized proteins. Sera always displayed strong IgE reactivity towards the wild-type allergen, reduced IgE binding to Mut-CD or Mut-EF and no binding to Mut-CD/EF.

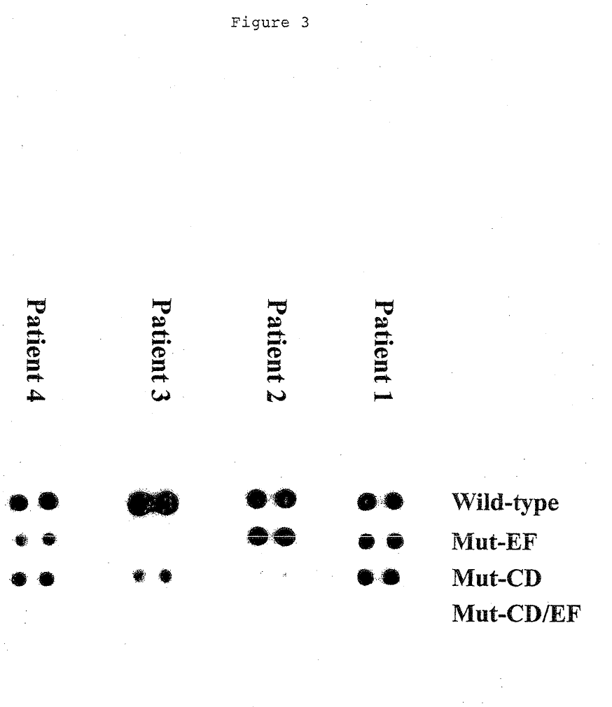

Furthermore, the inventors also evaluated the IgE binding capacity of the non-denatured, dot-blotted parvalbumin proteins using sera from 23 fish allergic patients (FIG. 3). Dot blot assays confirmed the data obtained in the immunoblot experiments and showed that modifications in one of the calcium binding domains (Mut-CD or Mut-EF) resulted in variable changes of IgE reactivity depending on the sera used. Some patients even displayed higher IgE reactivities to Mut-CD or Mut-EF than to the wild-type allergen. Mutations in both calcium-binding regions (Mut-CD/EF), on the other hand, caused a complete loss of IgE binding capacity in all of the sera tested. In case of Mut-CD/EF, quantification of the IgE reactivity by gamma counting revealed IgE binding capacities between 0.5% to 67.6% of the IgE binding capacities of parvalbumin wild-type protein (Table 3).

TABLE 3
IgE reactivity of fish allergic patients to dot-blotted recombinant
parvalbumin (wild-type) and to the parvalbumin mutants. IgE reactivity
was quantified by gamma counting. Results are displayed in counts
per minute (cpm) SEQ ID NO: 2) mutCD) and SEQ ID NO: 3 (mutEF)
are used to generate Table 3.

| Patient | Wild-type | Mut-EF | Mut-CD | Mut-CD/EF |
|---|---|---|---|---|
| 1 | 65.9 | 61.6 | 8.6 | 2.6 |
| 2 | 16.2 | 33.3 | 13.7 | 8.6 |
| 3 | 82.5 | 85.5 | 26.0 | 0.6 |
| 4 | 28.9 | 106.0 | 21.9 | 8.6 |
| 5 | 62.0 | 31.3 | 47.5 | 9.6 |
| 6 | 92.2 | 23.2 | 26.0 | 24.5 |
| 7 | 33.7 | 29.3 | 14.8 | 4.6 |
| 8 | 72.7 | 60.6 | 87.5 | 4.6 |
| 9 | 329.2 | 193.9 | 128.4 | 1.6 |
| 10 | 194.6 | 104.0 | 48.5 | 12.6 |
| 11 | 438.4 | 374.6 | 113.1 | 1.4 |
| 12 | 75.7 | 138.3 | 21.9 | 1.6 |
| 13 | 258.0 | 206.0 | 110.0 | 4.6 |
| 14 | 172.2 | 62.6 | 12.7 | 12.6 |
| 15 | 192.7 | 344.3 | 60.8 | 5.6 |
| 16 | 211.2 | 75.7 | 131.5 | 6.6 |
| 17 | 751.8 | 882.1 | 123.5 | 10.8 |
| 18 | 738.2 | 464.0 | 496.0 | 46.6 |
| 19 | 1442.8 | 45.2 | 165.2 | 17.1 |
| 20 | 473.4 | 169.6 | 208.1 | 19.0 |
| 21 | 84.8 | 89.4 | 70.1 | 41.5 |

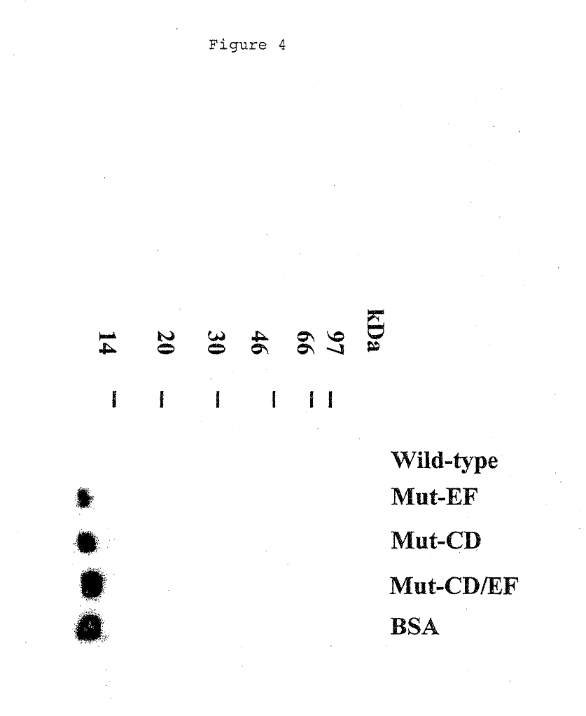

The inventors also investigated the capacity of the recombinant parvalbumin variants to inhibit IgE binding to nitrocellulose-blotted natural carp parvalbumin using immunoblot competition assays (FIG. 4). In these experiments patients sera were preadsorbed to an excess of the wild-type or the mutant proteins before exposure to the immobilised natural allergen. During preincubation reaction between proteins and IgE antibodies thus occurred in solution and thereby mimicked the in vivo situation. As shown in FIG. 4 IgE binding to the natural allergen was completely inhibited by addition of recombinant wild-type protein. Mut-CD and Mut-EF exhibited a markedly lower inhibition capacity than the wild-type protein, whereas preincubation with Mut-CD/EF had no effect on the IgE binding to natural parvalbumin.

These results suggested that by amino acid substitutions in either of the calcium binding sites the overall structure of the parvalbumin molecule was not significantly altered. Nevertheless, it can also be concluded that mutations in one of the domains did have an effect on local B cell epitopes, because Mut-CD or Mut-EF caused remarkable reduction of IgE reactivity in some of the sera. However, modifications in both calcium binding regions caused such a significant change of conformational epitopes and/or unfolding of the protein that IgE binding was completely abolished in all of the allergic patients sera tested.

Experimental Protocols:

Immunoblot and Dot Blot Analysis:

Reactivities of recombinant carp parvalbumin variants to serum IgE from fish allergic patients were determined in immunoblot or dot blot experiments. For immunoblots, 1.5 μg of the purified proteins were separated by SDS-PAGE (Laemmli 1970) and blotted onto nitrocellulose membranes (Schleicher&Schuell, Dassel, Germany; Towbin et al., 1979). For dot blot analysis, aliquots of approximately 0.5 μg of the parvalbumin proteins were dotted on nitrocellulose membranes (Schleicher&Schuell). In both cases filters were probed with patients sera diluted 1:10 in PBST (PBS, pH 7.5, containing 0.5% v/v Tween 20) and bound IgE antibodies were detected with 1:15 diluted ¹²⁵I-labelled anti-human IgE antibodies (RAST, Pharmacia, Uppsala, Sweden).

Immunoblot Competition Experiments:

For immunoblot inhibition experiments, sera from fish-allergic patients were preincubated with purified recombinant proteins and, for control purposes, with an immunologically unrelated protein (BSA) (10 μg/ml of 1:10 diluted serum). Thereafter, nitrocellulose-blotted purified natural parvalbumin was incubated with the preadsorbed serum samples and bound IgE was detected using ¹²⁵I-rabbit anti-human IgE (Pharmacia).

### Example 6

### Reduced Ability of Recombinant Parvalbumin Mutants to Induce Basophil Histamine Release

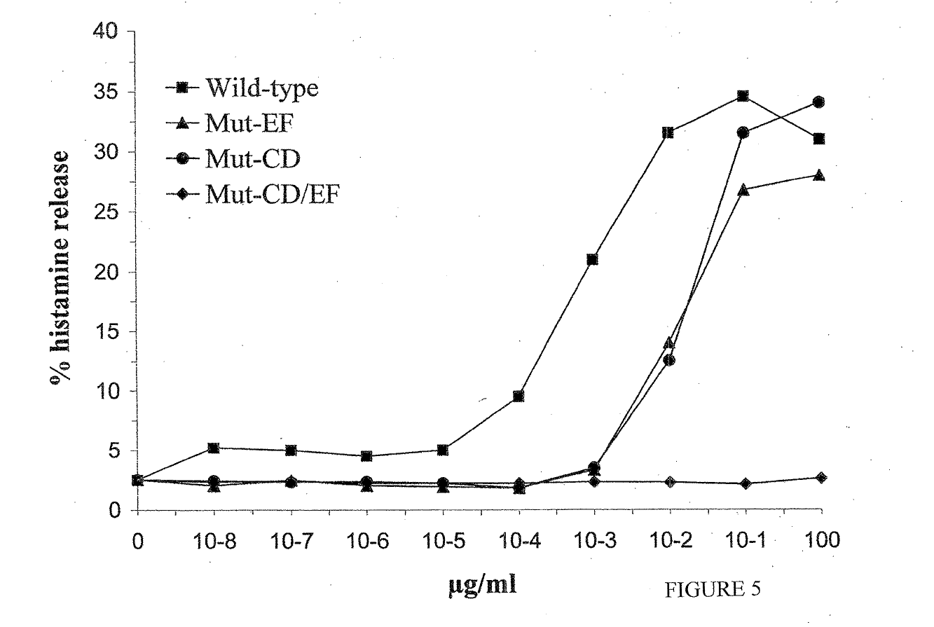

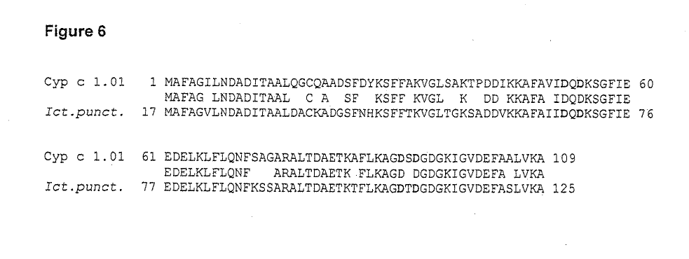

To analyse the biological activity of the parvalbumin mutants, peripheral blood basophils of a fish allergic patient were incubated with different concentrations of the recombinant wild-type and mutant proteins (FIG. 5). A strong dose-dependent release of histamine from the basophil granulocytes was induced by the recombinant wild-type protein. Amounts of released histamine were less, if granulocytes were exposed to Mut-CD or Mut-EF and highly reduced after exposure of the cells to Mut-CD/EF. This significantly reduced allergenic activity of Mut-CD/EF suggests that the mutated parvalbumin derivative represents a suitable candidate molecule for therapeutic applications.

Experimental Protocol:

Basophil Histamine Release Assay:

Granulocytes were isolated from heparinised blood samples of a fish allergic patient by dextran sedimentation. Cells were incubated with increasing concentrations of recombinant carp parvalbumin variants, anti-human IgE antibody, or buffer as previously described (Valent et al., 1989). Liberated histamine was measured in the cell-free supernatants by radioimmunoassay (Immunotech, Marseille, France).

### Example 7

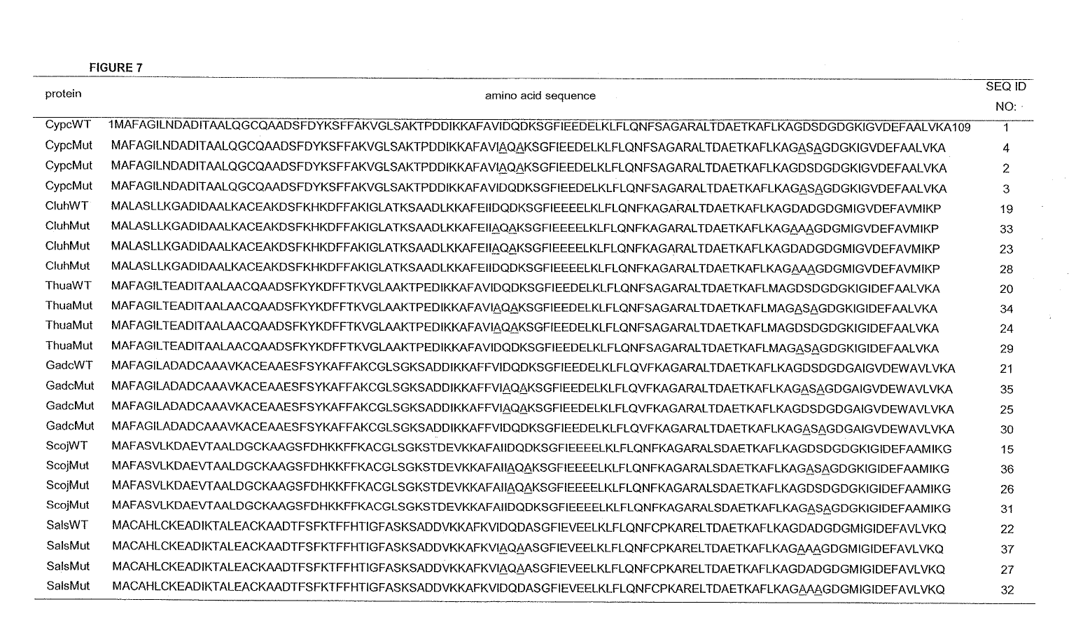

The aim of the following experiments was to confirm that the introduction of point mutations at amino acids 52, 54, 91 and 93 of fish parvalbumins, as described above, can be regarded as a general strategy for the development of hypoallergenic parvalbumin derivatives. To address this question, parvalbumins from Atlantic salmon (Sal s 1; accession number Q91482.1; van Do et al., 1999), from tuna (Thu a 1; accession number FM178217.1), from chub mackerel (Sco j 1; accession number P59747; Hamada et al., 2003, from herring (Clu h 1; accession number FM178220) and from codfish (Gad c 1; accession number AY035584; van Do et al., 2003) were selected and amino acids were exchanged at the specified positions aspartic acids (D) by alanines (A) (FIG. 7). For this, DNA sequences encoding wildtype as well as mutant parvalbumins were codon optimized for expression in Escherichia coli and synthesized by GenScript (Piscataway, N.J.). The synthetic genes were cloned into the NdeI and EcoRI restriction sites of the bacterial expression vector pET-27b (Novagen, Madison, Wis.). Expression of the recombinant proteins was induced in the E. coli strain BL21(DE3) by addition of IPTG (Swoboda et al., 2002 and 2007) and IgE reactivity of the proteins was evaluated by immunoblot analysis (Swoboda et al., 2002 and 2007). For this, bacterial protein extracts from induced E. coli cultures were separated by SDS-PAGE (Laemmli, 1970) and blotted onto nitrocellulose membranes (Towbin et al., 1979) that were exposed to sera from fish allergic patients. Bound IgE antibodies were detected with ¹²⁵I-labelled anti-human IgE antibodies (Swoboda et al., 2002 and 2007). For control purposes, bacterial protein extracts from E. coli cultures expressing the carp Cyp c 1 wildtype (Swoboda et al., 2002) and the Cyp c 1 mutant (Swoboda et al., 2007) protein were also included in the immunoblot experiments.

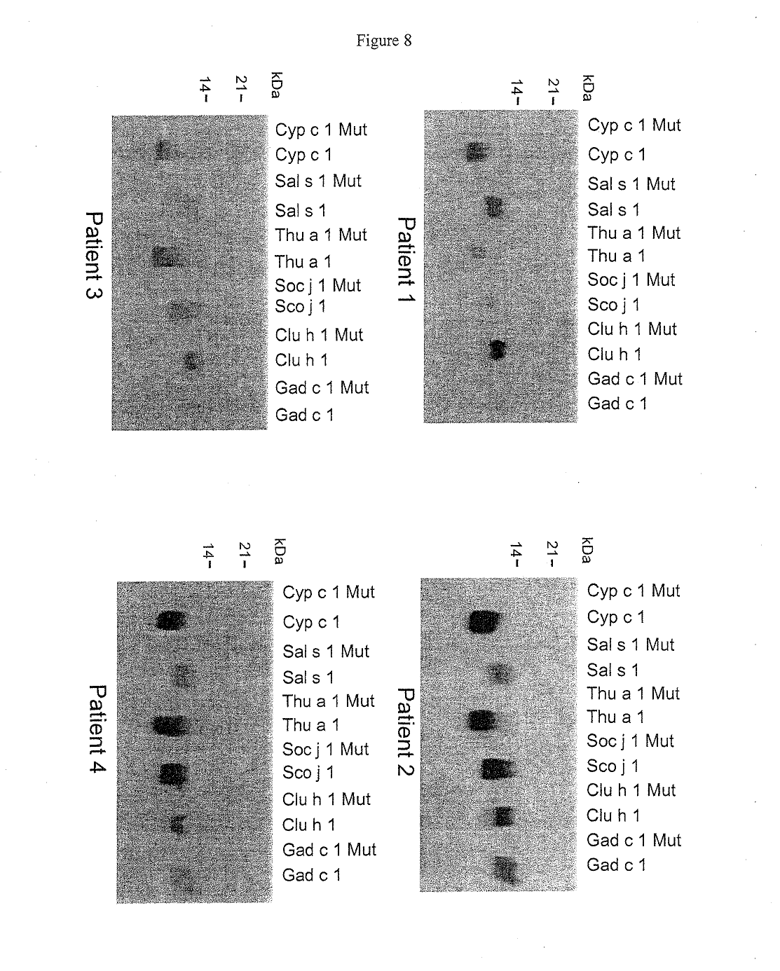

As exemplified in FIG. 8 with sera from four fish allergic patients, wildtype parvalbumins from the various fish species always displayed IgE reactivity, whereas none of the mutated variants could be recognized by patients' IgE. These results demonstrate that the exchange of aspartic acids by non-native amino acids (alanines) at the critical positions, specified in the patent, represents a general strategy for the generation of hypoallergenic fish parvalbumins.

### REFERENCES

Aas, K. 1987. Fish allergy and the cod allergen model. In Food allergy and intolerance, J. Brostoff, and S. T. Challacombe, eds. Balliere Tindall, London, p. 356.
Aas, K., and S. M. Elsayed. 1969. Characterisation of a major allergen (cod). J. Allergy 44:333.
Berchtold, M. W. 1989. Structure and expression of genes encoding three-domain Ca²⁺-binding proteins parvalbumin and oncomodulin. Biochim. Biophys. Acta 1009:201.
Bischoff, S. C., A. Herrmann, and M. P. Manns. 1996. Prevalence of adverse reactions to food in patients with gastrointestinal diseases. Allergy 51:811,
Bousquet, J., R. Lockey, H. J. Mailing, and the WHO panel members. 1998. Allergen immunotherapy: therapeutic vaccines for allergic diseases. J. Allergy Clin. Immunol. 102.558.
Bugajsaka-Schretter, A., L. Elfman, T. Fuchs, S. Kapiotis, H. Rumpold, R. Valenta, and S. Spitzauer. 1998. Parvalbumin, a cross-reactive fish allergen, contains IgE-binding epitopes sensitive to periodate treatment and Ca²⁺ depletion. J. Allergy Clin. Immunol. 101:67.
Bugajska-Schretter, A., M. Grote, L. Vangelista, P. Valent, W. R. Sperr, H. Rumpold, A. Pastore, R. Reichelt, R. Valenta, and S. Spitzauer. 2000. Purification, biochemical, and immunological characterisation of a major food allergen: different immunoglobulin E recognition of the apo- and calcium-bound forms of carp parvalbumin. Gut. 46:661.
De Martino, M., E. Novembre, L. Galli, A. De Marco, P. Botarelli, E. Marano, and A. Vierucci. 1990. Allergy to different fish species in cod-allergic children: in vivo and in vitro studies. J. Allergy Clin. Immunol. 86:909.
Declercq, J. P., B. Tinant, J. Parello, and J. Rambaud. 1991. Ionic interactions with parvalbumins. Crystal structure determination of pike 4.10 parvalbumin in four different ionic environments. J. Mol. Biol. 220:1017.
Etesamifar, M., and B. Wüthrich. 1998. IgE-mediated food allergies including oral allergy syndrome in 383 patients. Allergologie 21:451.
Elsayed, S., and K. Aas. 1971. Characterisation of a major allergen (cod). Observation on effect of denaturation on the allergenic activity. J. Allergy 47:283.
Etherton et al., Solid Phase Peptide Synthesis: A Practical Approach, IRL Press, Oxford 1989.
Goodman, M., and J. F. Pechere. 1977. The evolution of muscular parvalbumins investigated by the maximum parsimony method. J. Mol. Evol. 9:131.
Hamada Y, Tanaka H, Ishizaki S, Ishida M, Nagashima Y, Shiomi K. 2003. Purification, reactivity with IgE and cDNA cloning of parvalbumin as the major allergen of mackerels. Food Chem Toxicol 41(8):1149-56.
Heizmann, C. W., and W. Hunziker. 1991. Intracellular calcium-binding proteins: more sites than insights. Trends Biochem. Sci. 16:98.
Ikura, M. 1996. Calcium-binding and conformation response in EF-hand proteins. Trends Biochem. Sci. 21:14.
Kretsinger, R. H., and C. E. Nockold. 1973. Carp muscle calcium-binding protein. IL Structure determination and general description. J. Biol. Chem. 248:3313.
Kretsinger, R. H. 1980. Structure and evolution of calcium-modulated proteins. C. R. C. Crit. Rev. Biochem. 8:119.
Laemmli U K. 1970. Cleavage of structural proteins during the assembly of the head of bacteriophage T4. Nature 227(5259):680-5.
Lehky, P., H. E. Blum, E. A. Stein, and E. H. Fisher. 1974. Isolation and characterisation of parvalbumins from skeletal muscle of higher vertebrates. J. Biol. Chem. 249:4332.
Lindstroem, C. D. V., T. van Do, L Hordvik, C. Endresen, and S. Elsayed. 1996. Cloning of two distinct cDNAs encoding parvalbumin, the major allergen of Atlantic salmon (Salmo salar). Scand. J. Immunol. 44:335.
Merrifield et al. 1963. J. Am. Chem. Soc. 85:2149.
Niederberger et al. 1998. J. Allergy Clin. Immunol. 102:579.
O′Neil, C., A. A. Helbling, and S. B. Lehrer. 1993. Allergic reactions to fish. Clin. Rev. Allergy 11:183.
Pascual, C., M. M. Esteban, and J. F. Crespo. 1992. Fish allergy: evaluation of the importance of cross-reactivity. J. Pediatr. 121:29.
Pauli et al. 2000. Clin. Exp. Allergy 30:1076.
Pechere, J. F. 1997. The significance of parvalbumin among muscular calcium proteins. In Calcium-Binding Proteins and Calcium. R. H. Wasserman, R. Corradino, E. Carafoli, R. H. Kretsinger, D. H. MacLennan, and F. L. Siegel, eds. Elsevier, Holland, p. 213.
Permyakov, E. A., V. N. Medvedkin, Y. V. Mitin, and R. H. Kretsinger. 1991. Noncovalent complex between domain AB and domain CD*EF of parvalbumn. Biochim, Biiophys. Acta 1076:667.
Remington: The Science and Practice of Pharmacy, Gennaro, Ed., Mack Publishing Co., Easton, Pa., 19th Edition 1995.
Sambrook et al., Molecular Cloning, a Laboratory Manual, Cold Spring Harbor Laboratory, 1989.
Sampson, H. 1999. Food allergy. Part 1: Immunopathogenesis and clinical disorders. J. Allergy Clin. Immunol. 103:717.
Sanger, F., S. Nicklen, and A. R. Coulson. 1977. DNA sequencing with chain terminating. Proc. Natl. Acad, Sci. USA 74:5463.
Scopes, Protein Purification. Springer Verlag, Heidelberg 1994.
Swoboda I, Bugajska-Schretter A, Verdino P, Keller W, Sperr W R, Valent P, Valenta R, Spitzauer S. 2002. Recombinant carp parvalbumin, the major cross-reactive fish allergen: a tool for diagnosis and therapy of fish allergy. J Immunol 168(9):4576-84.
Swoboda I, Bugajska-Schretter A, Linhart B, Verdino P, Keller W, Schulmeister U, Sperr W R, Valent P, Peltre G, Quirce S, Douladiris N, Papadopoulos N G, Valenta R, Spitzauer S. 2007. A recombinant hypoallergenic parvalbumin mutant for immunotherapy of IgE-mediated fish allergy. J Immunol 178(10):6290-6.
Tatusova et al. 1999. FEMS Microbiol. Lett. 174:247.
Towbin, H., T. Staehelin, and J. Gordon. 1979. Electrophoretic transfer of proteins from polyacrylamide gels to nitrocellulose sheets: procedure and some applications. Proc. Natl. Acad. Sci, USA 76:4350.
Valent, P., J. Besemer, M. Muhm, O. Majdic, K. Lechner, and P. Bettelheim. 1989. Interleukin 3 activates human blood basophils via high-affinity binding sites. Proc. Natl. Acad. Sci. USA 86:5542.
Valenta, R., and D. Kraft. 1995. Recombinant allergens for diagnosis and therapy of allergic diseases. Curr. Opin. Immunol. 7:751.
Valenta, R., S. Vrtala, S. Laffer, S. Spitzauer, and D. Kraft. 1998. Recombinant allergens. Allergy 53:552.
Van Do T, Hordvik I, Endresen C, Elsayed S. 1999. Expression and analysis of recombinant salmon parvalbumin, the major allergen in Atlantic salmon (Salmo salar). Scand J Immunol 50(6):619-25.
Van Do T, Hordvik I, Endresen C, Elsayed S. 2003. The major allergen (parvalbumin) of codfish is encoded by at least two isotypic genes: cDNA cloning, expression and antibody binding of the recombinant allergens. Mol Immunol 39(10):595-602.
van Hage-Hamsten et al. 1999. J. Allergy Clin. Immunol. 104:969.
Vrtala et al. 1997. J. Clin. Invest. 99:1673.
Methods in Enzymology, Vol. 182, Guide to Protein Purification, Academic Press New York, 1990.

1. An isolated fish parvalbumin polypeptide comprising an amino acid sequence that is at least 60% identical to SEQ ID NO:1 and which upon alignment of said amino acid sequence with SEQ ID NO:1, the amino acid(s) in at least two of the positions that correspond to positions 52, 54, 91 and 93 of SEQ ID NO:1, differs from the amino acids at such positions in the native amino acid sequence of said polypeptide, and wherein said fish parvalbumin polypeptide has a reduced allergenic activity compared to SEQ ID NO:1.

2-17. (canceled)


SEQUENCE LISTING (38 sequences)

SEQ ID NO: 1 - protein, 109 aa, Cyprinus carpio
MAFAGILNDADITAALQGCQAADSFDYKSFFAKVGLSAKTPDDIKKAFAVIDQDKSGFIEEDELKLFLQNFSAGARALTDAETKAFLKAGDSDGDGKIGVDEFAALVKA

SEQ ID NO: 2 - protein, 109 aa, Artificial Sequence
  Description of Artificial Sequence Synthetic polypeptide
MAFAGILNDADITAALQGCQAADSFDYKSFFAKVGLSAKTPDDIKKAFAVIAQAKSGFIEEDELKLFLQNFSAGARALTDAETKAFLKAGDSDGDGKIGVDEFAALVKA

SEQ ID NO: 3 - protein, 109 aa, Artificial Sequence
  Description of Artificial Sequence Synthetic polypeptide
MAFAGILNDADITAALQGCQAADSFDYKSFFAKVGLSAKTPDDIKKAFAVIDQDKSGFIEEDELKLFLQNFSAGARALTDAETKAFLKAGASAGDGKIGVDEFAALVKA

SEQ ID NO: 4 - protein, 109 aa, Artificial Sequence
  Description of Artificial Sequence Synthetic polypeptide
MAFAGILNDADITAALQGCQAADSFDYKSFFAKVGLSAKTPDDIKKAFAVIAQAKSGFIEEDELKLFLQNFSAGARALTDAETKAFLKAGASAGDGKIGVDEFAALVKA

SEQ ID NO: 5 - protein, 62 aa, Artificial Sequence
  Description of Artificial Sequence Synthetic polypeptide
AFAGILNDADITAALQGCQAADSFDYKSFFAKVGLSAKTPDDIKKAFAVIDQDKSGFIEEDE

SEQ ID NO: 6 - protein, 19 aa, Artificial Sequence
  Description of Artificial Sequence Synthetic polypeptide
DSDGDGKIGVDEFAALVKA

SEQ ID NO: 7 - protein, 109 aa, Danio rerio
MAFAGILNEADITAALQACQAADSFDYKSFFAKVGLSAKTPDDIKKAFAVIDQDKSGFIEEDELKLFLQNFSAGARALTDAETKAFLKAGDSDGDGKIGVDEFASLVKA

SEQ ID NO: 8 - protein, 108 aa, Cyprinus carpio
  MOD_RES (19)..(19): Any amino acid
AYGGILNDADITAALEACXAXDSFNAKSFFAKVGLSAKTPDDIKKAFAVIDQDKSGFIEEDELKLFLQNFSAGARALTDAETKAFLKAGDSDGDGKIGVDEFAALVKA

SEQ ID NO: 9 - protein, 109 aa, Cyprinus carpio
MAFAGVLNDADITAALEACKAADSFNHKTFFAKVGLTSKSADDVKKAFAIIDQDKSGFIEEDELKLFLQNFKAGARALTDGETKTFLKAGDSDGDGKIGVDEFTALVKA

SEQ ID NO: 10 - protein, 125 aa, Ictalurus punctatus
VFENLQLTTKKTKELKMAFAGVLNDADITAALDACKADGSFNHKSFFTKVGLTGKSADDVKKAFAIIDQDKSGFIEEDELKLFLQNFKSSARALTDAETKTFLKAGDTDGDGKIGVDEFASLVKA

SEQ ID NO: 11 - protein, 109 aa, Gadus morhua
MAFAGILNDADITAALAACKAEGSFDHKAFFTKVGLAAKSPADIKKVFEIIDQDKSDFVEEDELKLFLQNFSAGARALSDAETKVFLKAGDSDGDGKIGVDEFGAMIKA

SEQ ID NO: 12 - protein, 109 aa, Danio rerio
MAFAGILKDEDVAAALKDCAAADSFNYKNFFAKVGLSAKSPDDIKKAFFVIDQDKSGFIEEDELKLFLQNFSAGARALTDAETKAFLSAGDSDGDGKIGVDEFALLVKA

SEQ ID NO: 13 - protein, 109 aa, Danio rerio
MAFAGVLNDADISAALEACKAADSFNHKSFFAKVGLASKSADEVKKAFAIIDQDKSGFIEEEELKLFLQNFKADARALTDGETKTFLKAGDSYGDGKIGIYEFAAFVKA

SEQ ID NO: 14 - protein, 108 aa, Electrophorus electricus
AFAGVLNDADITAALDACKADGSFDHKAFFEKVGLTSKSADDVKKAFAIIDQDKSGFIEEDELKLFLQNFKSGARALTDAETKAFMKAGDTDGDGKIGVEEFSALVKA

SEQ ID NO: 15 - protein, 109 aa, Scomber japonicus
MAFASVLKDAEVTAALDGCKAAGSFDHKKFFKACGLSGKSTDEVKKAFAIIDQDKSGFIEEEELKLFLQNFKAGARALSDAETKAFLKAGDSDGDGKIGIDEFAAMIKG

SEQ ID NO: 16 - DNA, 45 bp, Artificial Sequence
  Description of Artificial Sequence Synthetic primer
aaggcctttgctgtcattgcccaagccaagagcggcttcattgag

SEQ ID NO: 17 - DNA, 45 bp, Artificial Sequence
  Description of Artificial Sequence Synthetic primer
gccttcctgaaagctggagcctctgctggtgatggcaagattgga

SEQ ID NO: 18 - DNA, 330 bp, Cyprinus carpio
atggcattcgctggaattctgaatgatgctgacatcactgcagccctgcagggctgccaagctgctgactccttcgactacaagagcttcttcgccaaggttggtctgtctgccaagactcctgatgacatcaagaaggcctttgctgtcattgaccaagacaagagcggcttcattgaggaggatgagctgaaactgttcctgcagaacttctctgctggcgccagggcactcactgatgcagagacaaaggccttcctgaaagctggagactctgatggtgatggcaagattggagttgatgagtttgctgccctggtcaaggcataa

SEQ ID NO: 19 - protein, 109 aa, Clupea harengus
MALASLLKGADIDAALKACEAKDSFKHKDFFAKIGLATKSAADLKKAFEIIDQDKSGFIEEEELKLFLQNFKAGARALTDAETKAFLKAGDADGDGMIGVDEFAVMIKP

SEQ ID NO: 20 - protein, 109 aa, Thunnus albacares
MAFAGILTEADITAALAACQAADSFKYKDFFTKVGLAAKTPEDIKKAFAVIDQDKSGFIEEDELKLFLQNFSAGARALTDAETKAFLMAGDSDGDGKIGIDEFAALVKA

SEQ ID NO: 21 - protein, 109 aa, Gadus morhua
MAFAGILADADCAAAVKACEAAESFSYKAFFAKCGLSGKSADDIKKAFFVIDQDKSGFIEEDELKLFLQVFKAGARALTDAETKAFLKAGDSDGDGAIGVDEWAVLVKA

SEQ ID NO: 22 - protein, 109 aa, Salmo salar
MACAHLCKEADIKTALEACKAADTFSFKTFFHTIGFASKSADDVKKAFKVIDQDASGFIEVEELKLFLQNFCPKARELTDAETKAFLKAGDADGDGMIGIDEFAVLVKQ

SEQ ID NO: 23 - protein, 109 aa, Artificial Sequence
  Description of Artificial Sequence Synthetic polypeptide
MALASLLKGADIDAALKACEAKDSFKHKDFFAKIGLATKSAADLKKAFEIIAQAKSGFIEEEELKLFLQNFKAGARALTDAETKAFLKAGDADGDGMIGVDEFAVMIKP

SEQ ID NO: 24 - protein, 109 aa, Artificial Sequence
  Description of Artificial Sequence Synthetic polypeptide
MAFAGILTEADITAALAACQAADSFKYKDFFTKVGLAAKTPEDIKKAFAVIAQAKSGFIEEDELKLFLQNFSAGARALTDAETKAFLMAGDSDGDGKIGIDEFAALVKA

SEQ ID NO: 25 - protein, 109 aa, Artificial Sequence
  Description of Artificial Sequence Synthetic polypeptide
MAFAGILADADCAAAVKACEAAESFSYKAFFAKCGLSGKSADDIKKAFFVIAQAKSGFIEEDELKLFLQVFKAGARALTDAETKAFLKAGDSDGDGAIGVDEWAVLVKA

SEQ ID NO: 26 - protein, 109 aa, Artificial Sequence
  Description of Artificial Sequence Synthetic polypeptide
MAFASVLKDAEVTAALDGCKAAGSFDHKKFFKACGLSGKSTDEVKKAFAIIAQAKSGFIEEEELKLFLQNFKAGARALSDAETKAFLKAGDSDGDGKIGIDEFAAMIKG

SEQ ID NO: 27 - protein, 109 aa, Artificial Sequence
  Description of Artificial Sequence Synthetic polypeptide
MACAHLCKEADIKTALEACKAADTFSFKTFFHTIGFASKSADDVKKAFKVIAQAASGFIEVEELKLFLQNFCPKARELTDAETKAFLKAGDADGDGMIGIDEFAVLVKQ

SEQ ID NO: 28 - protein, 109 aa, Artificial Sequence
  Description of Artificial Sequence Synthetic polypeptide
MALASLLKGADIDAALKACEAKDSFKHKDFFAKIGLATKSAADLKKAFEIIDQDKSGFIEEEELKLFLQNFKAGARALTDAETKAFLKAGAAAGDGMIGVDEFAVMIKP

SEQ ID NO: 29 - protein, 109 aa, Artificial Sequence
  Description of Artificial Sequence Synthetic polypeptide
MAFAGILTEADITAALAACQAADSFKYKDFFTKVGLAAKTPEDIKKAFAVIDQDKSGFIEEDELKLFLQNFSAGARALTDAETKAFLMAGASAGDGKIGIDEFAALVKA

SEQ ID NO: 30 - protein, 109 aa, Artificial Sequence
  Description of Artificial Sequence Synthetic polypeptide
MAFAGILADADCAAAVKACEAAESFSYKAFFAKCGLSGKSADDIKKAFFVIDQDKSGFIEEDELKLFLQVFKAGARALTDAETKAFLKAGASAGDGAIGVDEWAVLVKA

SEQ ID NO: 31 - protein, 109 aa, Artificial Sequence
  Description of Artificial Sequence Synthetic polypeptide
MAFASVLKDAEVTAALDGCKAAGSFDHKKFFKACGLSGKSTDEVKKAFAIIDQDKSGFIEEEELKLFLQNFKAGARALSDAETKAFLKAGASAGDGKIGIDEFAAMIKG

SEQ ID NO: 32 - protein, 109 aa, Artificial Sequence
  Description of Artificial Sequence Synthetic polypeptide
MACAHLCKEADIKTALEACKAADTFSFKTFFHTIGFASKSADDVKKAFKVIDQDASGFIEVEELKLFLQNFCPKARELTDAETKAFLKAGAAAGDGMIGIDEFAVLVKQ

SEQ ID NO: 33 - protein, 109 aa, Artificial Sequence
  Description of Artificial Sequence Synthetic polypeptide
MALASLLKGADIDAALKACEAKDSFKHKDFFAKIGLATKSAADLKKAFEIIAQAKSGFIEEEELKLFLQNFKAGARALTDAETKAFLKAGAAAGDGMIGVDEFAVMIKP

SEQ ID NO: 34 - protein, 109 aa, Artificial Sequence
  Description of Artificial Sequence Synthetic polypeptide
MAFAGILTEADITAALAACQAADSFKYKDFFTKVGLAAKTPEDIKKAFAVIAQAKSGFIEEDELKLFLQNFSAGARALTDAETKAFLMAGASAGDGKIGIDEFAALVKA

SEQ ID NO: 35 - protein, 109 aa, Artificial Sequence
  Description of Artificial Sequence Synthetic polypeptide
MAFAGILADADCAAAVKACEAAESFSYKAFFAKCGLSGKSADDIKKAFFVIAQAKSGFIEEDELKLFLQVFKAGARALTDAETKAFLKAGASAGDGAIGVDEWAVLVKA

SEQ ID NO: 36 - protein, 109 aa, Artificial Sequence
  Description of Artificial Sequence Synthetic polypeptide
MAFASVLKDAEVTAALDGCKAAGSFDHKKFFKACGLSGKSTDEVKKAFAIIAQAKSGFIEEEELKLFLQNFKAGARALSDAETKAFLKAGASAGDGKIGIDEFAAMIKG

SEQ ID NO: 37 - protein, 109 aa, Artificial Sequence
  Description of Artificial Sequence Synthetic polypeptide
MACAHLCKEADIKTALEACKAADTFSFKTFFHTIGFASKSADDVKKAFKVIAQAASGFIEVEELKLFLQNFCPKARELTDAETKAFLKAGAAAGDGMIGIDEFAVLVKQ

SEQ ID NO: 38 - protein, 6 aa, Artificial Sequence
  Description of Artificial Sequence Synthetic 6xHis tag
HHHHHH